(19) United States
(12) Patent Application Publication
(10) Pub. No.: US 2013/0237891 A1
(43) Pub. Date: Sep. 12, 2013

(54) ORTHOTIC BRACE TIGHTENING DEVICE

(71) Applicants: Marshall Fryman, Anaheim, CA (US); Thomas H. Grimm, Napa, CA (US)

(72) Inventors: Marshall Fryman, Anaheim, CA (US); Thomas H. Grimm, Napa, CA (US)

(21) Appl. No.: 13/603,772

(22) Filed: Sep. 5, 2012

Related U.S. Application Data
(60) Provisional application No. 61/531,724, filed on Sep. 7, 2011.

(51) Int. Cl. A61F 5/02

(52) U.S. Cl. CPC A61F 5/02
USPC 602/19

(57) ABSTRACT

The invention is a back brace with a stacked pulley tightening device that allows a user to quickly, efficiently, and easily tighten the back brace around the user after the back brace is donned. The pulleys provide a mechanical advantage. Additionally, the present invention includes a segmented belt portion such that a single pulley tightening device can be used to tighten around users of any size.

### CROSS-REFERENCE TO RELATED APPLICATIONS

This Non-Provisional Patent Application claims priority to Provisional Application No. 61/531,724, titled “ORTHOTIC BRACE TIGHTENING DEVICE,” filed Sep. 7, 2011, by Marshall Fryman and Thomas H. Grimm, the entire contents of which are hereby incorporated by this reference as though set forth in its entirety.

### FIELD OF THE INVENTION

The invention relates to a device, method, and system for tightening an orthotic brace. More particularly, the invention is an orthotic back brace with a pulley tightening device and a segmented belt for easy size readjustments.

### BACKGROUND OF THE INVENTION

Orthotic braces have been manufactured for centuries for medical purposes. An orthotic or orthosis is an orthopedic device that supports or corrects the function of a limb or other part of the body. An orthopedic brace, also referred to as an appliance or brace, is an orthopedic device that is typically used to control, guide, assist, limit, and/or immobilize an extremity, joint, or body segment for a particular reason.

Orthotic back braces are generally used to limit the mobility of the wearer for a particular reason, which is often medically-related. Such braces date to the 18th century. They have been used not only to correct spinal curvature problems but also to support the spine in order to relieve stress, especially when performing physical activities such as lifting heavy objects.

A common problem in traditional back braces is the amount of effort required to tighten the device properly. Recently, advances have been made in stress-relieving back braces that allows a user to tighten the brace around the wearer after the brace is donned. Unfortunately, the typical mechanism for tightening a back brace after the brace is donned (often springs or elastic) requires a significant amount of effort and physical strength to operate. The enormous strength and effort required in the prior art devices show that there is a need in the art for a mechanically-advantaged back brace tightening device.

Regarding back braces that disclose a pulley system, which tightens a back brace around a wearer, U.S. Pat. No. 6,213,968, issued to Heinz (“Heinz”) discloses such a device. There are, however, significant deficiencies in Heinz. For example, the Heinz reference uses a side-by-side pulley, which does not take advantage of a stacked configuration that includes a user cord and a separate compression cord. Accordingly, the Heinz reference, although mechanically advantaged, does not use the greater advantage of a stacked pulley. Without the stacked pulley, the user still has to use significant force to tighten the back brace. Additionally, the Heinz brace provides no protection for the user from being pinched by the two segments tightening together. Finally, the Heinz reference fails to include a segmented belt portion, which permits the back brace to adjust the fitting for wearers of all sizes.

Thus, what is needed is a back brace tightening device that provides a stacked pulley system, a pulley cover pad, and a segmented belt portion. The present invention provides such a back brace tightening device.

### SUMMARY OF THE INVENTION

To minimize the limitations in the prior art, and to minimize other limitations that will become apparent upon reading and understanding the present specification, the present invention discloses a new and useful device, method, and system for tightening a back brace around a wearer that includes a stacked pulley system.

One embodiment of the invention is an orthotic tightening device comprising: one or more main pulley housings; one or more secondary housings; one or more compression cords; one or more user cords; and one or more stacked pulleys. The stacked pulleys are within one of the one or more main pulley housings. Each of the one or more stacked pulleys has at least a first pulley and a second pulley, wherein the first and second pulleys are combined in a stacked configuration. Each of the one or more user cords are attached to the first pulley. Each of the one or more compression cords are attached to the second pulley, pass through one of the one or more secondary housings, and are secured to the same main pulley housing in which the second pulley, to which the compression cord is attached, is located. When a user pulls the one or more user cords, the one or more main pulley housings and the one or more secondary housings are drawn toward each other. Preferably the first pulley has a diameter that is greater than a diameter of the second pulley, such that a mechanical advantage is gained when the one or more user cords are pulled such that the one or more main pulley housings and the one or more secondary housings are drawn toward each other. Preferably, the orthotic tightening device may also include one or more panels; and an orthotic device. The orthotic device is preferably donned by the user and the panels are preferably attached to the orthotic device. The main pulley housings and the secondary housings are attached to one of the panels, such that when the user cords are pulled and the main pulley housings and the secondary housings are drawn toward each other, the orthotic device is tightened around the user wearing the orthotic device. Preferably the orthotic device is a back brace that includes a lumbar pad and a plurality of side portions. Preferably, the plurality of side portions are comprised of one or more segments and the side portions are configured to wrap around the user and the segments detachable connect to each other. Preferably, there are at least two segments on each of the plurality of side portions, such that the user is able to adjust a length of the plurality of side portions to fit a plurality of differently sized users. Preferably there are one or more handles, which are attached to the one or more user cords, such that the user may use the one or more handles to pull on the one or more user cords. Preferably, there are two panels, two main pulley housings, two secondary housings, two user cords, two compression cords, two handles, and two side portions. Preferably, the two side portions form a belt of the back brace and the two panels are each attached to a different side portion of the two side portions, such that the two panels are facing each other in parallel manner. The two panels preferably include one of the two main pulley housings and one of the two secondary housings such that each of the two main pulley housings is directly across from a corresponding secondary housing, such that two pulley sets are formed with a first pulley set above a second pulley set, such that each of the two pulley sets tighten independently of each other. Preferably, each of the two pulley sets includes one compression cord and one user cord. Preferably, the two handles detachably engage with the side portions after the user has tightened the tightening device, such that the tightening device is kept in a tightened state until the user loosens the tightening device. The back brace may also include a cover, wherein the cover is positioned over the tightening device to protect the user and the tightening device.

Another embodiment of the invention is an orthotic back brace with a stacked pulley tightening device comprising: a lumbar pad; two side portions; and a tightening device. The tightening device is comprised of a plurality of pulley sets, wherein each of the plurality of pulley sets is comprised of a main pulley housing, a secondary housing, a user cord, and a compression cord, such that there are a plurality of main pulley housings, a plurality of secondary housings, a plurality of user cords, and a plurality of compression cords. Each of the plurality of main pulley housings has a stacked pulley, such that there is a plurality of stacked pulleys. Each of the plurality of stacked pulleys has at least a first pulley and a second pulley, such that there are plurality of first pulleys and a plurality of second pulleys. Each of the plurality of first and second pulleys are combined in a stacked configuration. Each of the plurality of user cords are attached to one of the plurality of first pulleys. Each of the plurality of compression cords are attached to one of the plurality of second pulleys, pass through one of the at least one secondary housings, and are secured to the same main pulley housing in which the second pulley is located. When a user pulls the plurality of user cords, the plurality of main pulley housings and the plurality of secondary housings are drawn toward each other. Preferably, the plurality of first pulleys have a diameter that is greater than a diameter of the plurality of second pulleys, such that a mechanical advantage is gained when the plurality of user cords are pulled such that the plurality of main pulley housings and the plurality of secondary housings are drawn toward each other. Preferably, there are two pulley sets. Preferably, the tightening device is further comprised of two panels. Preferably, the two panels are each attached to a different side portion of the two side portions, such that the two panels are facing each other in parallel manner. Each of the two panels includes one of the two main pulley housings and one of the two secondary housings such that each of the two main pulley housings is directly across from a corresponding secondary housing, such that the two pulley sets are positioned with a first pulley set above a second pulley set, such that each of the two pulley sets tightens independently of the other. Preferably, the two side portions form a belt of the back brace and the back brace is donned by the user. Preferably, the plurality of side portions are comprised of at least two segments. Preferably, the side portions are configured to wrap around the user and detachable connect to each other when donned by the user. Preferably, the segments on each of the two side portions allows the user to adjust a length of the plurality of side portions to fit a plurality of differently sized users, and preferably they detachable connect to one another. Preferably the back brace may include a plurality of handles; wherein the plurality of handles are attached to the plurality of user cords, such that the user may use the plurality of handles to pull on the plurality of user cords. Preferably, the plurality of handles detachably engage with the side portions after the user has tightened the tightening device, such that the tightening device is kept in a tightened state until the user loosens the tightening device. The back brace may also include a cover; wherein the cover is positioned over the tightening device. Preferably, the invention is a back brace with a pulley tightening device that allows a user to quickly, efficiently, and easily tighten the back brace around the user after the back brace is donned. The pulleys provide a mechanical advantage. Additionally, the present invention preferably includes a segmented belt portion such that a single pulley tightening device can be used to tighten around nearly any sized individual.

Another embodiment of the invention is a stacked pulley tightening device for a brace comprising: at least two sets of pulleys and a brace device. The two sets of pulleys are comprised of: a first pulley set; and a second pulley set. The first pulley set and the second pulley set are positioned vertically relative to each other. The first and second pulley sets are comprised of a stacked pulley housing, a secondary housing, a compression cord, and a user cord. Preferably, the stacked pulley housing is comprised of a stacked pulley. The stacked pulley is comprised of a small diameter pulley and a large diameter pulley. The compression cord is wrapped around the small diameter pulley and the user cord is wrapped around the large diameter pulley. The two independent pulley systems allow a wearer to control the tightness of the top and bottom of the brace separately. Further, this stacked pulley system provides a significant mechanical advantage not found in the prior art.

Additionally, one embodiment of the belt portion of the present invention includes segmented side portions so that the belt can be increased or decreased in size to accommodate a variety of different human trunk girths.

Another embodiment of the present invention is a tightening system for an orthotic brace that comprises a belt that is divided into at least two segments, a left side and a right side. The brace features an adjustable tightening mechanism that permits the wearer to further adjust the brace by means of increasing or decreasing the distance between the left and right sides at the lumbar area. Once the desired tightness is achieved, the wearer can affix the handles (that are attached to the user cord) to the belt portion via a hook and loop fastener means.

The tightening mechanism preferably consists of at least two sets of pulleys manufactured so that the outer wall of one pulley is permanently affixed to the outer wall of the next pulley in sequence. Pulleys that are permanently attached to each other or machined as a single piece are known in the art as “stacked pulleys.” The stacked pulley system offers a number of advantages over non-stacked pulley systems. First, because they are able to vary the size of the pulley from one pulley in the sequence to the next, there is a mechanical advantage that is gained permitting force multiplication. By altering the relative pulley sizes, many desired force multiplications are possible. By stacking additional pulleys beyond two, it is even possible to achieve multiple sets of force multiplication from the same originating force. By altering the mechanical advantage, it is possible to achieve closure (or tightening) with less cordage and effort than with non-mechanical advantage systems. Second, because the cord, string or other winding material is not connected from one pulley to the next, it is possible to precisely control the amount of closure the wearer is able to achieve. In competing systems, the winding material is typically directly connected to the closure permitting the wearer to tighten the closure even to the point of injury.

An alternative embodiment of the invention is an apparatus that allows the wearer to easily tighten a donned orthotic back brace. The tightening mechanism is mounted onto a lumbar pad and two back/side panels, which extend around the wearer's torso. The tightening mechanism consists of two sets of pulleys, vertically positioned as a top set and a bottom set. Each of the two sets of pulleys has a left bank and a right bank. Centered between the two banks is a force multiplying two stage pulley, or stacked pulley. The stacked pulley is comprised of a smaller radius pulley and a larger radius pulley. Although the large pulley and the small pulley turn, spin, or rotate concurrently, they are connected to two different cords. A user cord is connected to a handle that is pulled by the wearer on one end and terminates fixed to the large pulley on the opposite end. The compression cord originates as fixed to the small pulley then engages the two banks of pulleys and terminates in a connection with the one of the two banks. When the wearer pulls the handles, the two stacked pulleys are turned and the compression cords retract into the small pulley and pull the two banks of each of the pulley sets together. This action tightens the back brace around the wearer.

It is an object of the present invention to permit a back brace wearer to have a mechanical advantage in tightening her brace so that the brace provides more secure support for the back and does not require the extreme force required in conventional back braces.

It is an object of the present invention to have a back brace with a segmented belt portion to provide a tightening mechanism on various different length belts.

It is an object of the present invention to overcome the limitations of the prior art.

These, as well as other components, steps, features, objects, benefits, and advantages, will now become clear from a review of the following detailed description of illustrative embodiments, the accompanying drawings, and the claims.

### BRIEF DESCRIPTION OF THE DRAWINGS

The drawings are of illustrative embodiments. They do not illustrate all embodiments. Other embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

FIG. 1 is an illustration of an exterior side of one embodiment of the back brace.

FIG. 2 is an illustration of an interior side of one embodiment of the back brace.

FIG. 3 is an illustration of a front view of an external side of one embodiment of the back brace and shows the back brace when secured.

FIG. 4 is an illustration of an external side of one embodiment of the tightening device of the back brace and shows the back brace without the back pad cover.

FIG. 5 is an illustration of one embodiment of the back brace with the tightening device and shows the pulleys removed from the pulley housings.

FIG. 6 is a detailed illustration of the secondary pulley housing of one embodiment of the tightening device.

FIG. 7 is a detailed illustration of the primary pulley housing of one embodiment of the tightening device.

FIG. 8 is a detailed illustration of one embodiment of the tightening device and shows the user and compression cords.

FIG. 9 is a detailed illustration of one embodiment of the tightening device and shows how the compression cord and user cord connect to the stacked pulley.

FIG. 10 is an illustration of an alternative embodiment of the tightening device.

### DETAILED DESCRIPTION OF THE DRAWINGS

The drawings are of illustrative embodiments. They do not illustrate all embodiments. Other embodiments or variations of embodiments may be used in addition or instead. Details which may be apparent or unnecessary may be omitted to save space or for more effective illustration. Some embodiments may be practiced with additional components or steps and/or without all of the components or steps which are illustrated. When the same numeral appears in different drawings, it refers to the same or like components or steps.

In the following detailed description of various embodiments of the invention, numerous specific details are set forth in order to provide a thorough understanding of various aspects of one or more embodiments of the invention. However, one or more embodiments of the invention may be practiced without some or all of these specific details. In other instances, well-known methods, procedures, and/or components have not been described in detail so as not to unnecessarily obscure aspects of embodiments of the invention.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the Drawings, and the detailed descriptions thereof, are to be regarded as illustrative in nature and not restrictive. Also, the reference or non-reference to a particular embodiment of the invention shall not be interpreted to limit the scope of the invention.

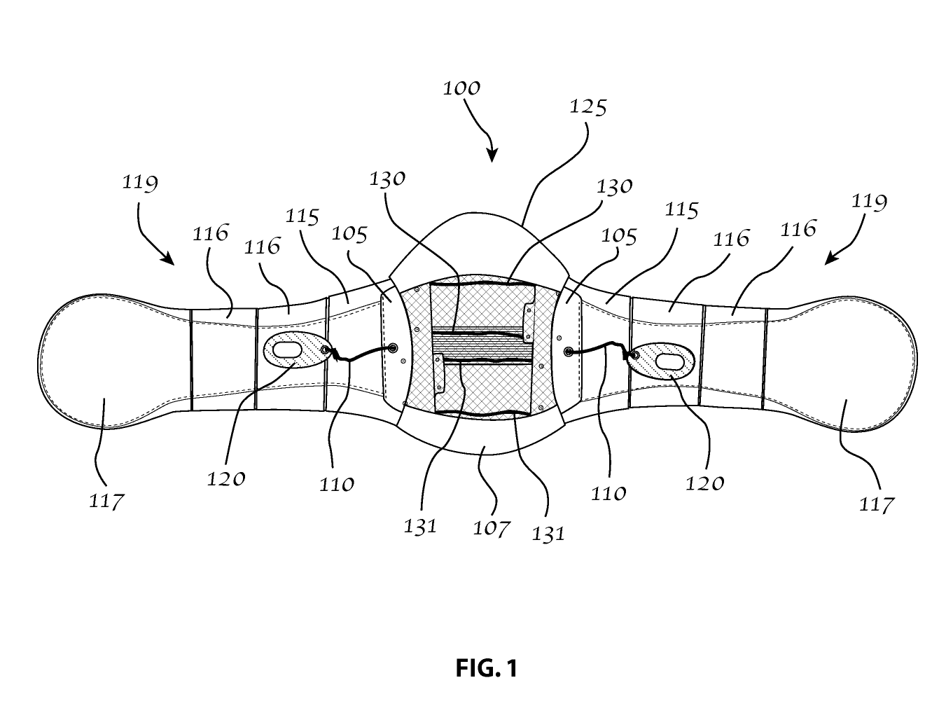

FIG. 1 is an illustration of an exterior side of one embodiment of the back brace. As shown in FIG. 1, one embodiment of the back brace 100 includes: a tightening device 105; cover 107; segments 115, 116, 117; belt side portions 119; handles 120; lumbar support pad 125; and compression cords 130, 131.The tightening device 105 is preferably attached to the back brace 100 at belt side portions 119. The belt side portions 119 are preferably segmented into at least segments 115, 116, and 117 to allow wearers of various girths to adjust the length of back brace 100. The length is preferably adjustable by adding or removing segment 116. Although any securing device may connect segments 115, 116, and 117, these segments are preferably secured together using hook and loop or hook and eye fasteners. The tightening device 105 is preferably covered by a cover 107. The cover 107 is preferably made from a synthetic mesh fabric—but may be made out of any durable material—and preferably prevents a wearer or other person from easily accessing or damaging the tightening device 105. The cover 107 preferably keeps the tightening mechanism 105 from being damaged or interfered with and may also limit the distance, in which the tightening device 105 can be loosened or pulled apart. Also depicted is the lumbar support pad 125.

FIG. 1 also shows how, in one embodiment, the two user cords 110 have handles 120 that secure to the side portions 119. The handles 120 are preferably covered with hook and loop (one side hook, the other side loop) material that allow them to fasten to the two side portions 119.

The handles 120 that are attached to the user cords 110 preferably make pulling the user cords 110 easier. The handle 120 may have a hole intended for a finger or thumb for ease of pulling. As shown in FIG. 1, the handles 120 are substantially flat, which allows them to be stored out of the way of the wearer when not being used to tighten or loosen the back brace.

The plurality of segments 115, 116, 117 typically allows back brace 100 to have a one-size-fits-all functionality. In addition to adding or removing segments, the segments themselves may be of various sizes, such as small, medium, large, and extra-large, in order to further increase the variability of the back brace 105. Alternatively, the back brace 100 can be made of a single main structure, tailored for a person of a specific size. Also pictured in FIG. 1 are compression cords 130 and 131.

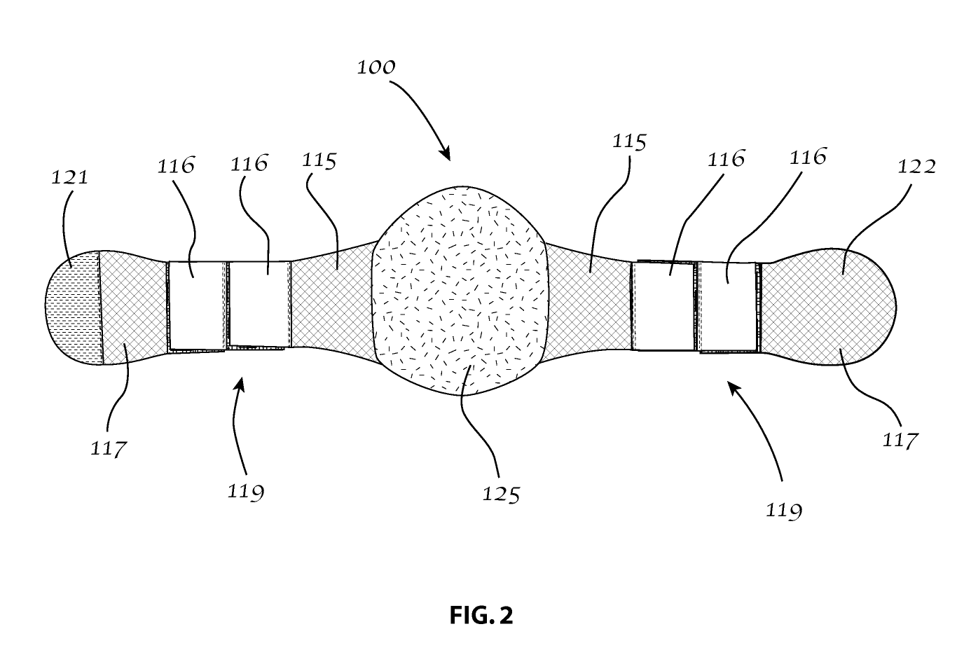

FIG. 2 is an illustration of an interior side of one embodiment of the back brace. As shown in FIG. 2, back brace 100 preferably includes: segments 115, 116, 117, belt side portions 119; handles 120; back brace ends 121, 122; lumbar support pad 125; and compression cords 130, 131. FIG. 2 shows the lumbar pad 125, which preferably provides orthotic support to the wearer and typically cushions the back against the tightening device. The lumbar pad 125 is preferably braced and cushioned for comfort. The lumbar pad 125 is also preferably elongated to support higher points on the back. Lumbar pad 125 may be removably attached to the side portions 119. The lumbar pad 125 is preferably removably attachable, via hook and loop material, to the inner surface of the side portions 119. Alternatively, the lumbar pad 125 may be permanently affixed to the side portions 119. The side portions 119 are preferably permanently attached to one another under the lumbar pad, but the two side portions 119 may be a single contiguous construction. Additionally, FIG. 2 provides a view of the opposite side from FIG. 1 of the segments 115, 116, and 117. The interior portion of the back brace 100 functions somewhat like a belt, and it makes contact with the wearer and is wrapped around the wearer's waist. Due to the functional tight fit of the brace 100 around the wearer, the interior has the potential to be uncomfortably warm. To minimize this, the interior of the side portions 119 and lumbar pad 125 may be lined with a mesh or breathable fabric that does not overly insulate and overheat the wearer. The end segments 117 preferably engage one another using hook and loop material at back brace ends 121, 122.

The entire belt potion, including the side portions 119, is, preferably, made of synthetic material because of its durability and strength. Alternatively, the entire belt may be made of any durable fabric, including any man-made, synthetic, or natural material.

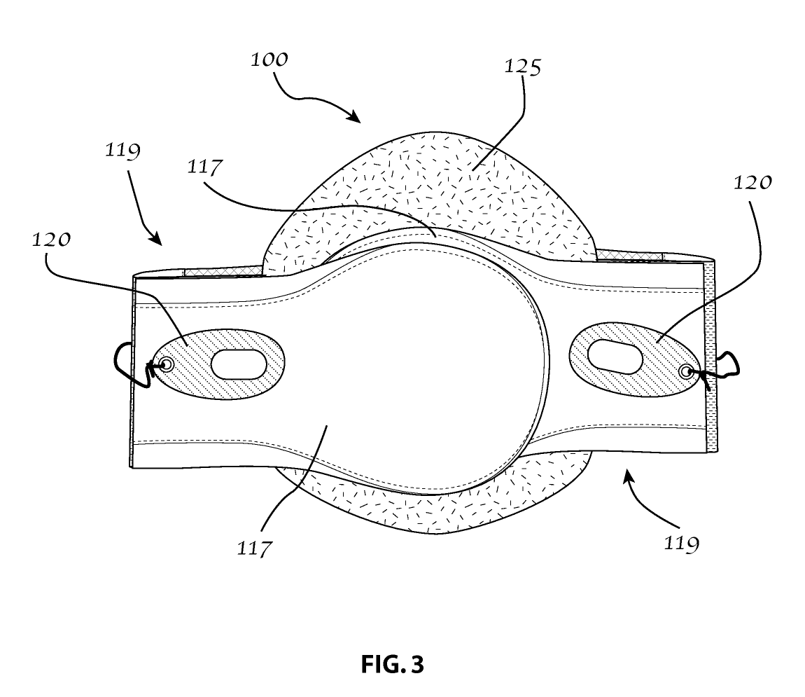

FIG. 3 is an illustration of a front view of an external side of one embodiment of the back brace and shows the back brace when secured. The back brace 100 is shown secured as it would likely fit around the wearer. The wearer's back would be in contact with the lumbar support 125 and the two end segments 117 preferably rest in front of the wearer and connected via a fastening device, which is preferably hook and loop material. Further, FIG. 3 shows the left and right segments of the belt portion 117 removably secured together preferably using hook and loop material. The belt side portions are alternatively referred to as “brace arms.” Additionally, the user cord handles 120 are shown engaged to the belt side portions 119.

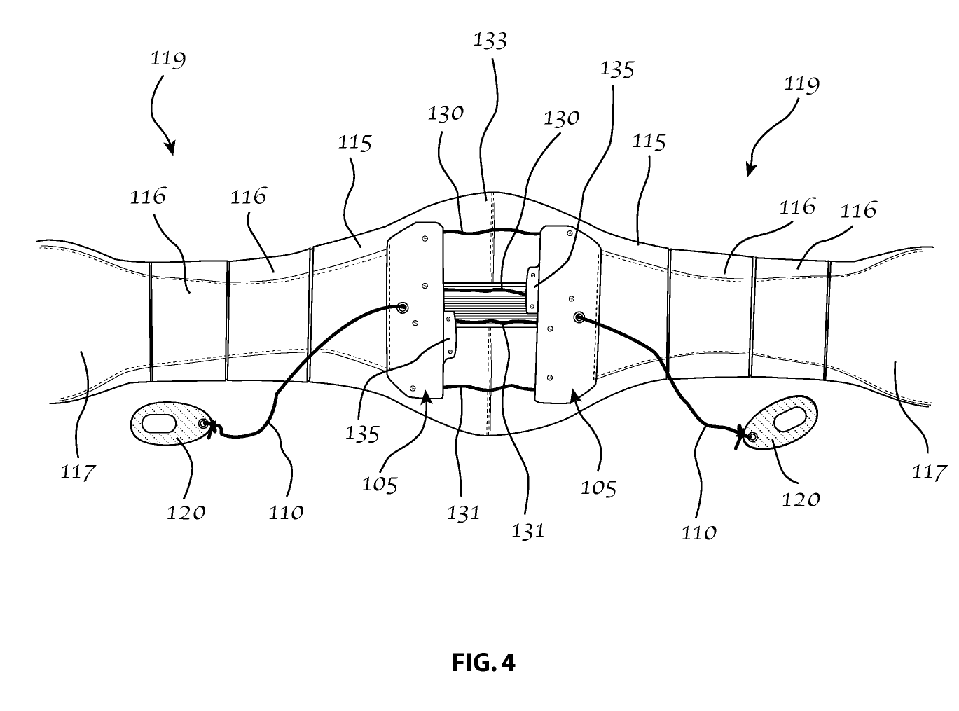

FIG. 4 is an illustration of an external side of one embodiment of the tightening device of the back brace and shows the back brace without the back pad cover. As shown in FIG. 4,back brace 100 preferably includes: tightening devices 105; user cords 110; handles 120; compression cords 130, 131; side portions 119; segments 115, 116, 117; and center segment 133. The tightening devices 105 are depicted uncovered by cover 107. When the tightening devices 105 are covered, as is preferred, the wearer (as well as people near the wearer) likely do not have easy access to the tightening devices 105. This likely prevents harm, such as pinching, to the wearer, those near the wearer as well as damage to the tightening device itself. The tightening device 105 is preferably a stacked pulley that, when the user handles 120 are pulled, the tightening devices 105 are pulled horizontally toward each other, causing the entire back brace to fit more securely, snuggly, and tightly, around the wearer. FIG. 4 shows that there are preferably two tightening devices 105, which permit the user to tighten the upper and lower portions of the back brace independently for an ideal fit. Further, side portions 119 are shown with the addition of extension segment 116 between segment 115 and end segment 117. The extension segment 116 preferably comes in a variety of lengths to accommodate any or all human girths.

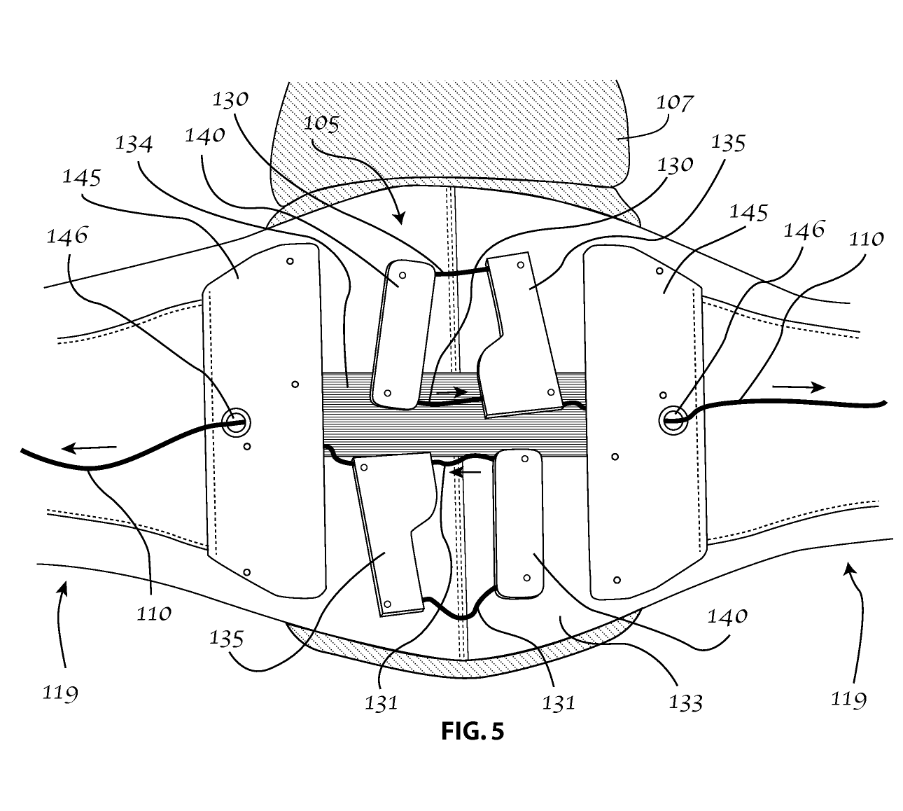

FIG. 5 is an illustration of one embodiment of the back brace with the tightening device and shows the pulleys removed from the pulley housings. FIG. 5 shows one embodiment of the tightening device 105, which preferably includes: main pulley housings 135; secondary housings 140; side panels 145; compression cords 130, 131; side panel openings 146; user cords 110; cover 107 (which is shown as removed); support strap 134; and center segment 133. Because the center segment 133 is preferably somewhat flexible, the support strap 134 prevents the center segment from being overly stretched when the tightening device 105 is pulled apart. FIG. 5 shows a preferred embodiment, wherein each side of the tightening device includes two side panels 145, which are secured to the side portions 119, and which are connected to and house the main pulley housings 135 and secondary housings 140. The main pulley housings 135 and the secondary housings 140 are typically secured to the side panels 145 via bolts or screws, but any connection or securing device may be used.

The panels 145 preferably includes top and bottom portions that sandwiches the pulley housings 135 and 140. The side panels 145 is preferably permanently connected to its side portion of the brace via stitching, but any connection device or mechanism may be use. Although not preferred, the panel may be absent and the pulley housings may be directly connected to the side portions 119 of the brace 100. Further shown are the user strings 110 and the compression strings 130 and 131. FIG. 5 shows how the preferably four (although there may be many more) pulley housings 135, 140 are attached to the pulley panels 145 to vertically align the two pulley housing sets. The two main pulley housings 135 are shown as slightly protruding from the panels 145 toward the opposite secondary housings 140.

FIG. 5 also shows how each set of pulley housings 135, 140 include one compression cord 130, 131, one user cord 110, one secondary housing 140, and one main pulley housing 135. The user cord 110 typically runs from the stacked pulley in the main stacked pulley housing 135 out through opening 146 in the panel 145. The arrows shown depict the direction of the cords 110, 130, 131 movement when a wearer is tightening the brace 100. The compression cords 130, 131 and the user strings 110 are preferably made of a relatively inelastic cord or string, such as nylon, but they may be made from any type of material. The main pulley housings 135 and secondary housings 140 are preferably made from high quality and durable plastic. Alternatively, the housings may be made of metal, composites, or other durable type of materials, without deviating from the scope of the invention.

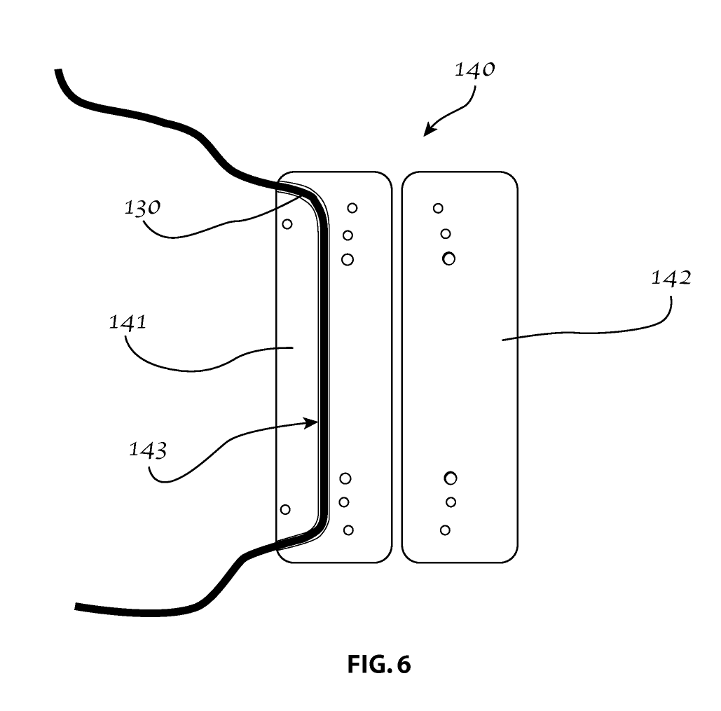

FIG. 6 is a detailed illustration of the secondary pulley housing of one embodiment of the tightening device. The secondary housing 140, as shown, is preferably comprised of two portions 141, 142. The string portion 141 typically has a compression cord channel 143 inside, which preferably allows the compression cord 130 to freely slide within with as little friction as possible. The string portion 141 is preferably secured to the top portion 142 using screws, pins, and bolts, or other connection means.

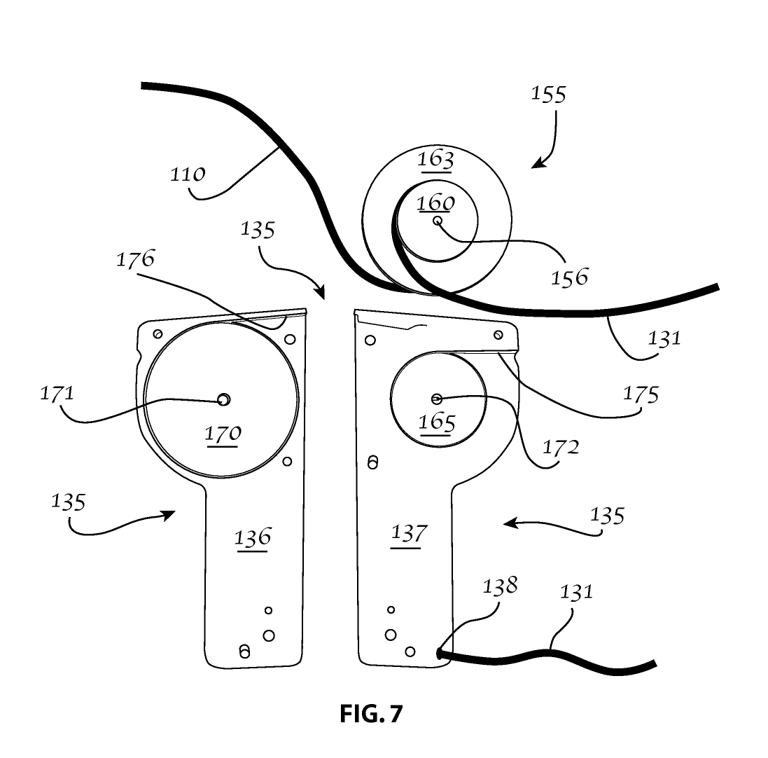

FIG. 7 is a detailed illustration of the primary pulley housing of one embodiment of the tightening device. The main pulley housings 135 are comprised of two sides 136, 137. Side 136 preferably has a fitted large pulley indentation 170 with an axle pin guide hole 171 in which one side of the pulley pin 156 preferably fits securely but rotatably. Additionally, side 136 preferably has a channel 176 in which the user cord 110 fits into and slides freely within with as little friction as possible.

Side 137 typically has a small pulley fitted indentation 165 to fit the small pulley 155. Side 137 typically also has an axle pin guide hole 172 in which the other side of the pulley pin 156 fits securely but rotatably. Side 137 typically also has a channel 175 in which one end of the compression cord 131 fits into and slides freely within.

FIG. 7 also shows the two tiered stacked pulley 155. The preferred ratio of radii of pulley 155 is 2:1 giving a preferred maximum of mechanical benefit of 2 times. In the preferred embodiment, the small radius pulley 160 (a second pulley) and the large radius pulley 163 (a first pulley) comprise as a single conjoined piece. The large pulley 163 is preferably attached to and functions in conjunction with the user cord 110. The small pulley 160 is preferably attached to and functions in conjunction with the compression cord 131. When both axel pins 156 are in their respective guide holes 171, 172, they function as an axel, permitting the stacked pulley 155 to freely rotate with minimal friction within main pulley housing 135 Importantly, the compression cord 131 should terminate securely at connection point 138. Compression cord 131 may be secured to point 138 by any connection device, including, but not limited to friction (via a knot), adhesive, or screw. The two pulley housing sides 136, 137 are secured together by bolts, screws, pins, or other securing devices.

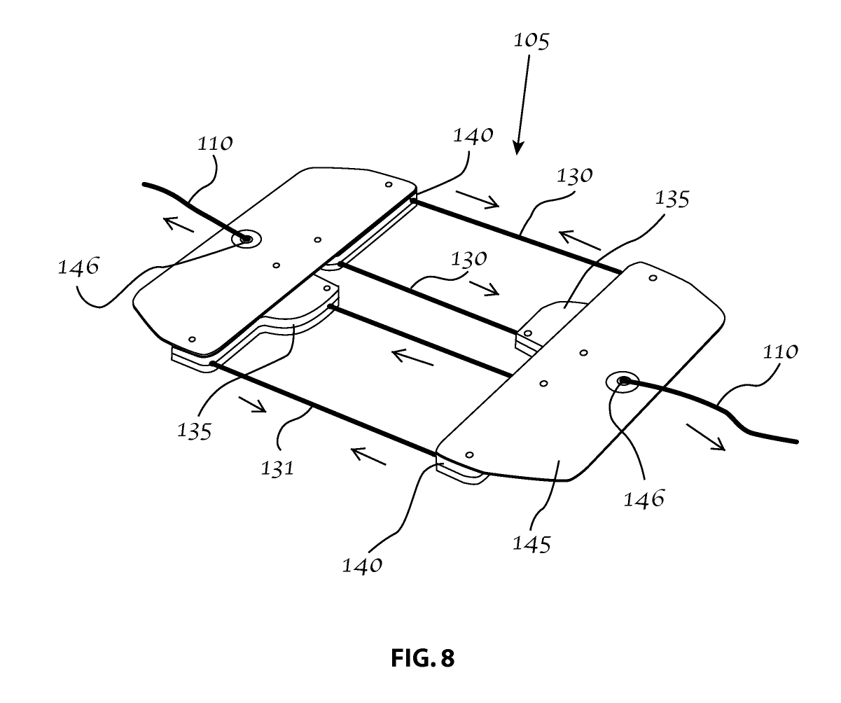

FIG. 8 is a detailed illustration of one embodiment of the tightening device and shows the user and compression cords. FIG. 8 shows the entire tightening device 105 separate from the back brace 100. Main pulley housings 135 and secondary housings 140 are fitted into opposite side panels 145, so that when the user strings 110 are pulled, the compression strings 130, 131 winds on the interior pulley 155 (not shown), and the two side panels 145 move closer to one another. As the side panels 145 move closer together, the brace 100 should be tightened, so that the brace 100 fits snuggly on the wearer. The arrows in FIG. 8 show movement of the cords and housings when the wearer pulls the user cords 110 Importantly, the two sets of main pulley housings 135 and secondary housings 140 typically permit a wearer to control the fit of the brace 100 at the top and bottom separately, providing the user an ideally fitted back brace 100. In the embodiment shown in FIG. 8, the right user cord 110 can control the bottom. and the left user cord 110 can control the top, or vice-versa. FIG. 8 shows the two side panels 145 in full extension, that is, as far away from one another that would likely be permitted by the cover and cords.

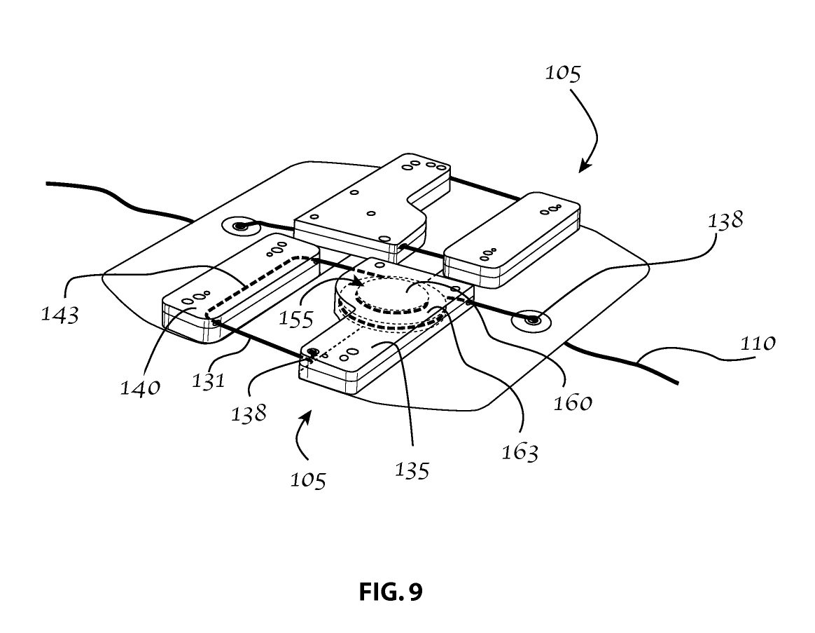

FIG. 9 is a detailed illustration of one embodiment of the tightening device and shows how the compression cord and user cord connect to the stacked pulley. FIG. 9 shows the tightening device 105, a transparent pulley housing 135, and transparent secondary housing 140 permitting the interaction of the cords and pulleys to be seen. The stacked pulley 155 is shown with the user string 110 engaging the large pulley 163, and the compression string 130 engaging the small pulley 160. The compression cord 131 preferably has a proximal end in the small pulley 160; preferably travels through the secondary housing 140 where the compression string goes through a compression string channel 143; preferably exits the secondary housing; and is typically fixed, by means of a knot or other connection device in point 138 on the main pulley housing 135. The compression cord 131 terminates in the stacked pulley housing 135, as preferred. The stacked pulley housing 135 typically has channels for the cords 110, 130, 131, so that they can slide into and out of the stacked pulley housing 135 to reach and attach to pulley 155. The pulley 155 typically turns within the stacked pulley housing 135, with as little friction as possible. When the tightening device 105 is tightened, the compression cord 131 preferably winds around the small diameter pulley 160, and the user cord 110 is unwound from the large diameter pulley 163. When the tightening device 105 is loosened, the user cord 110 preferably winds around the large diameter pulley 163, and the compression cord 131 unwinds from the small diameter pulley 160.

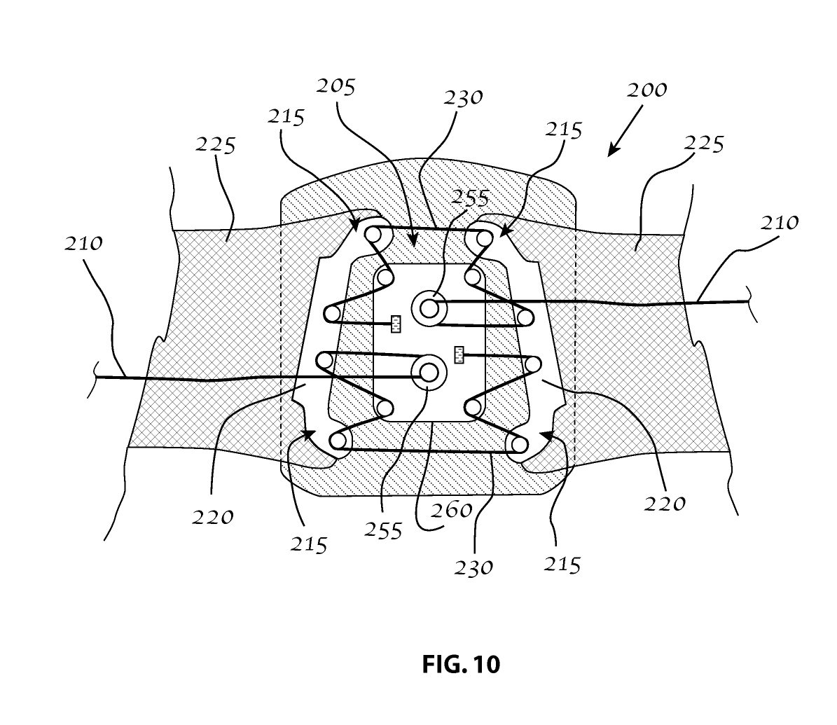

FIG. 10 is an illustration of an alternative embodiment of the tightening device. In the embodiment shown in FIG. 10, the entire back brace 200 and the tightening device 205 preferably comprise of two stacked pulleys 255, which are fixed to a center board 260. On two belt halves 225,stiffening members 220 are preferably attached. To these stiffening members 220, a plurality of pulleys 215 (preferably six) in one or two pulley banks are typically attached in a zig-zag pattern, as shown. This pattern permits the top and the bottom portions of the belt to be tightened or loosened independently of the other portion. This entire device 205 is on the lumbar section of the belt 200. This embodiment has compression cords 230 and a user cords 210.

Unless otherwise stated, all measurements, values, ratings, positions, magnitudes, sizes, locations, and other specifications which are set forth in this specification, including in the claims which follow, are approximate, not exact. They are intended to have a reasonable range which is consistent with the functions to which they relate and with what is customary in the art to which they pertain.

The foregoing description of the preferred embodiment of the invention has been presented for the purposes of illustration and description. While multiple embodiments are disclosed, still other embodiments of the present invention will become apparent to those skilled in the art from the above detailed description, which shows and describes illustrative embodiments of the invention. As will be realized, the invention is capable of modifications in various obvious aspects, all without departing from the spirit and scope of the present invention. Accordingly, the detailed description is to be regarded as illustrative in nature and not restrictive. Also, although not explicitly recited, one or more embodiments of the invention may be practiced in combination or conjunction with one another. Furthermore, the reference or non-reference to a particular embodiment of the invention shall not be interpreted to limit the scope the invention. It is intended that the scope of the invention not be limited by this detailed description, but by the claims and the equivalents to the claims that are appended hereto.

Except as stated immediately above, nothing which has been stated or illustrated is intended or should be interpreted to cause a dedication of any component, step, feature, object, benefit, advantage, or equivalent to the public, regardless of whether it is or is not recited in the claims.

What is claimed is:

1. An orthotic tightening device comprising:
one or more main pulley housings;
one or more secondary housings;
one or more compression cords;
one or more user cords; and
one or more stacked pulleys;
wherein each of said one or more stacked pulleys is within one of said one or more main pulley housings;
wherein each of said one or more stacked pulleys has at least a first pulley and a second pulley,
wherein said first pulley and said second pulley are combined in a stacked configuration;
wherein each of said one or more user cords are attached to said first pulley;
wherein each of said one or more compression cords are attached to said second pulley, pass through one of said one or more secondary housings, and are secured to the same main pulley housing; and
wherein when a user pulls said one or more user cords, said one or more main pulley housings and said one or more secondary housings are drawn toward each other.

2. The orthotic tightening device of claim 1, wherein said first pulley has a diameter that is greater than a diameter of said second pulley, such that a mechanical advantage is gained when said one or more user cords are pulled such that said one or more main pulley housings and said one or more secondary housings are drawn toward each other.

3. The orthotic tightening device of claim 2, further comprising:
one or more panels; and
an orthotic device;
wherein said orthotic device is donned by said user;
wherein said one or more panels are attached to said orthotic device;
wherein said one or more main pulley housings and said one or more secondary housings are attached to one of said one or more panels, such that when said one or more user cords are pulled and when said one or more main pulley housings and said one or more secondary housings are drawn toward each other, said orthotic device is tightened around said user wearing said orthotic device.

4. The orthotic tightening device of claim 3, wherein said orthotic device is a back brace;
wherein said back brace is comprised of a lumbar pad and a plurality of side portions;
wherein said plurality of side portions are comprised of one or more segments;
wherein said plurality of side portions are configured to wrap around said user and detachably connect to each other; and
wherein said one or more segments are configured to detachable connect to each other.

5. The orthotic tightening device of claim 4, wherein there are at least two segments on each of said plurality of side portions, such that said user is able to adjust a length of said plurality of side portions to fit a plurality of differently sized users.

6. The orthotic tightening device of claim 5, further comprising:
one or more handles;
wherein said one or more handles are attached to said one or more user cords, such that said user may use said one or more handles to pull on said one or more user cords.

7. The orthotic tightening device of claim 6, wherein there are two panels;
wherein there are two main pulley housings;
wherein there are two secondary housings;
wherein there are two user cords;
wherein there are two compression cords;
wherein there are two handles; and
wherein there are two side portions.

8. The orthotic tightening device of claim 7, wherein said two side portions form a belt of said back brace;
wherein said two panels are each attached to a different side portion of said two side portions, such that said two panels are facing each other in parallel manner;
wherein each of said two panels includes one of said two main pulley housings and one of said two secondary housings such that each of said two main pulley housings is directly across from a corresponding secondary housing, such that two pulley sets are formed with a first pulley set above a second pulley set, such that each of said two pulley sets tighten independently of each other.

9. The orthotic tightening device of claim 8, wherein each of said two pulley sets includes one compression cord and one user cord.

10. The orthotic tightening device of claim 9, wherein said two handles detachably engage with said side portions after said user has tightened said tightening device, such that said tightening device is kept in a tightened state until said user loosens said tightening device.

11. The back brace from claim 10, further comprising:
a cover;
wherein said cover is positioned over said tightening device.

12. An orthotic back brace with a stacked pulley tightening device comprising:
a lumbar pad;
two side portions; and
a tightening device;
wherein said tightening device is comprised of a plurality of pulley sets;
wherein each of said plurality of pulley sets is comprised of a main pulley housing, a secondary housing, a user cord, and a compression cord, such that there are a plurality of main pulley housings, a plurality of secondary housings, a plurality of user cords, and a plurality of compression cords; and
wherein each of said plurality of main pulley housings has a stacked pulley, such that there is a plurality of stacked pulleys.

13. The orthotic back brace of claim 12, wherein each of said plurality of stacked pulleys has at least a first pulley and a second pulley, such that there are plurality of first pulleys and a plurality of second pulleys;
wherein each of said plurality of first and second pulleys are combined in a stacked configuration;
wherein each of said plurality of user cords are attached to one of said plurality of first pulleys;
wherein each of said plurality of compression cords are attached to one of said plurality of second pulleys, pass through one of said at least one secondary housings, and are secured to the same main pulley housing in which said second pulley is located; and
wherein when a user pulls said plurality of user cords, said plurality of main pulley housings and said plurality of secondary housings are drawn toward each other.

14. The orthotic back brace of claim 13, wherein said plurality of first pulleys have a diameter that is greater than a diameter of said plurality of second pulleys, such that a mechanical advantage is gained when said plurality of user cords are pulled such that said plurality of main pulley housings and said plurality of secondary housings are drawn toward each other.

15. The orthotic back brace of claim 14, wherein there are two pulley sets;
wherein said tightening device is further comprised of two panels;
wherein said two panels are each attached to a different side portion of said two side portions, such that said two panels are facing each other in parallel manner; and
wherein each of said two panels includes one of said two main pulley housings and one of said two secondary housings such that each of said two main pulley housings is directly across from a corresponding secondary housing, such that said two pulley sets are positioned with a first pulley set above a second pulley set, such that each of said two pulley sets tightens independently of the other.

16. The orthotic back brace of claim 15, wherein said two side portions form a belt of said back brace;
wherein said back brace is donned by said user;
wherein said plurality of side portions are comprised of at least two segments;
wherein said plurality of side portions are configured to wrap around said user and detachably connect to each other when donned by said user; and
wherein said at least two segments are configured to detachable connect to each other.

17. The orthotic back brace of claim 16, wherein said at least two segments on each of said two side portions allows said user to adjust a length of said plurality of side portions to fit a plurality of differently sized users.

18. The orthotic back brace of claim 17, further comprising:
a plurality of handles;
wherein said plurality of handles are attached to said plurality of user cords, such that said user may use said plurality of handles to pull on said plurality of user cords.

19. The orthotic back brace of claim 18, wherein said plurality of handles detachably engage with said side portions after said user has tightened said tightening device, such that said tightening device is kept in a tightened state until said user loosens said tightening device.

20. The orthotic back brace from claim 19, further comprising:
a cover;
wherein said cover is positioned over said tightening device.
